SEQUENCE LISTING (2 sequences)

SEQ ID NO: 1 - DNA, 3215 bp, Homo sapiens
  CDS (150)..(1664)
gccgccccgccccgagaccgggcccgggggcgcggggcggcgggatgcggcgcccggggcggcgatgaccgcggagcgcacgccgcgggcccggccctgaccccgccgcccgcccgctgagccccccgccgaggtccggacaggccgagatgacgccgagccccctgttgctgctcctgctgccgccgctgctgctgggggccttcccgccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagcctgagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgccagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctagacggcaccgtatctgcagtgggcacgggggggccggccagacaggcagactgggaggatggaggacggagctgcagacgaaggcaggggacccatggcgaggaggaatggccagcaccccaggcagtctgtgtgtgaggcatagcccctggacacacacacacagacacacacactgcctggatgcatgtatgcacacacatgcgcgcacacgtgctccctgaaggcacacgtacgcacacacgcacatgcacagatatgccgcctgggcacacagataagctgcccaaatgcacgcacacgcacagagacatgccagaacatacaaggacatgctgcctgaacatacacacgcacacccatgcgcagatgtgctgcctggacacacacacacacacggatatgctgtctggacgcacacacgtgcagatatggtatccggacacacacgtgcacagatatgctgcctggacacacagataatgctgccttgacacacacatgcacggatattgcctggacacacacacacacacgtgtgcacagatatgctgtctggacacgcacacacatgcagatatgctgcctggacacacacttccagacacacgtgcacaggcgcagatatgctgcctggacacacgcagatatgctgtctagtcacacacacacgcagacatgctgtccggacacacacacgcatgcacagatatgctgtccggacacacacacgcacgcagatatgctgcctggacacacacacagataatgctgcctcaacactcacacacgtgcagatattgcctggacacacacatgtgcacagatatgctgtctggacatgcacacacgtgcagatatgctgtccggatacacacgcacgcacacatgcagatatgctgcctgggcacacacttccggacacacatgcacacacaggtgcagatatgctgcctggacacacgcagactgacgtgcttttgggagggtgtgccgtgaagcctgcagtacgtgtgccgtgaggctcatagttgatgagggactttccctgctccaccgtcactcccccaactctgcccgcctctgtccccgcctcagtccccgcctccatccccgcctctgtcccctggccttggcggctatttttgccacctgccttgggtgcccaggagtcccctactgctgtgggctggggttgggggcacagcagccccaagcctgagaggctggagcccatggctagtggctcatccccactgcattctccccctgacacagagaaggggccttggtatttatatttaagaaatgaagataatattaataatgatggaaggaagactgggttgcagggactgtggtctctcctggggcccgggacccgcctggtctttcagccatgctgatgaccacaccccgtccaggccagacaccaccccccaccccactgtcgtggtggccccagatctctgtaattttatgtagagtttgagctgaagccccgtatatttaatttattttgttaaacatgaaagtgcatcctttccctcca

SEQ ID NO: 2 - protein, 504 aa, Homo sapiens
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPPGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

(19) United States
(12) Patent Application Publication
(10) Pub. No.: US 2016/0326251 A1
(43) Pub. Date: Nov. 10, 2016

(54) THERAPEUTIC AGENT FOR CANCER, AND METHOD FOR DETERMINING PROGNOSIS OF CANCER

(71) Applicant: KYOTO UNIVERSITY, Kyoto-shi (JP)

(72) Inventors: Soken TSUCHIYA, Kyoto-shi (JP); Gozoh TSUJIMOTO, Kyoto-shi (JP); Kazuharu SHIMIZU, Kyoto-shi (JP); Yutaka SHIMADA, Toyama-shi (JP); Kazuhiro TSUKADA, Toyama-shi (JP)

(73) Assignee: KYOTO UNIVERSITY, Kyoto-shi (JP)

(21) Appl. No.: 15/222,660

(22) Filed: Jul. 28, 2016

Related U.S. Application Data
(63) Continuation of application No. 14/009,026, filed on Oct. 25, 2013, now abandoned.

(30) Foreign Application Priority Data
Mar. 31, 2011 (JP) 2011-080261

(51) Int. Cl. C07K 16/28; C07K 16/30

(52) U.S. Cl. CPC C07K 16/2863; C07K 16/30; C07K 2317/34; C07K 2317/73; C07K 2317/24

(57) ABSTRACT

Disclosed are a novel therapeutic agent for cancer such as esophageal squamous cell carcinoma, a method for predicting the prognosis of cancer, and a method for detecting, or predicting the prognosis of, cancer such as esophageal squamous cell carcinoma using a sample that can be collected less invasively. The therapeutic agent for cancer comprises as an effective component an antibody that undergoes antigen-antibody reaction with FGFRL1 to suppress the growth of cancer cells, or an antigen-binding fragment thereof. The method for predicting the prognosis of cancer comprises investigating the expression level of FGFRL1 in a cancer tissue separated from a living body, and, in this method, a high expression level of FGFRL1 indicates poor prognosis. The method for detecting cancer comprises measuring FGFRL1 or a fragment thereof extracted from a body tissue, or FGFRL1 or a fragment thereof in blood separated from a living body, and, in this method, a higher concentration of FGFRL1 or the fragment thereof contained therein than the concentration of FGFRL1 or the fragment thereof in the tissue or blood of a healthy individual indicates the presence of cancer.

### CROSS-REFERENCE TO RELATED APPLICATIONS

This application is a Continuation of co-pending application Ser. No. 14/009,026, filed on Oct. 25, 2013, which was filed as PCT International Application No. PCT/JP2012/058668 on Mar. 30, 2012, which claims the benefit under 35 U.S.C. §119(a) to Patent Application No. 2011-080261, filed in Japan on Mar. 31, 2011, all of which are hereby expressly incorporated by reference into the present application.

### REFERENCE TO SEQUENCE LISTING SUBMITTED VIA EFS-WEB

This application includes an electronically submitted sequence listing in .txt format. The .txt file contains a sequence listing entitled “2016-07-14_SEQ_LIST_0760-0437PUS1.txt.” created on Jul. 14, 2016 and is 14,076 bytes in size. The sequence listing contained in this .txt file is part of the specification and is hereby incorporated by reference herein in its entirety.

### TECHNICAL FIELD

The present invention relates to a therapeutic agent for cancer, a method for predicting the prognosis of cancer, and a method for detecting cancer.

### BACKGROUND ART

Esophagus cancer is a cancer with the eighth highest incidence rate and the sixth highest number of deaths. In Japan, esophageal squamous cell carcinoma (ESCC) accounts for not less than 90% of esophagus cancer. ESCC is a highly malignant cancer that frequently causes distant metastasis and recurrence, and its prognosis is generally poor.

On the other hand, it has been reported that ovarian cancer shows abnormal expression of fibroblast growth factor receptor like-1 (hereinafter referred to as “FGFRL1”) (Non-patent Document 1). However, in this report, no statistical analysis was carried out for the expression level of FGFRL1, and no analysis on the function of FGFRL1 was performed. Thus, this report never leads to inference of promotion of the cell growth by FGFRL1, utilization of its expression level for prediction of the prognosis, or its industrial applicability. Further, although it has been reported that microRNA (miRNA)-210 is involved in oncogenesis and that one of its target genes is FGFRL1 (Non-patent Document 2), this report does not clearly suggest utilization of FGFRL1 for prediction of the prognosis or for therapeutic agents. Further, the present inventors previously discovered that, in esophageal squamous cell carcinoma, microRNA-210 regulates the growth of cancer cells via FGFRL1 (Non-patent Document 3). However, this report only elucidated that a target gene of microRNA-210 is FGFRL1 and discussed about its downstream pathway, and no suggestion was made about possible use of an anti-FGFRL1 antibody for a therapeutic agent for cancer or a tool for prediction of the prognosis.

On the other hand, although FGFRL1 has been named a “molecule like a fibroblast growth factor receptor (FGFR)”, it is clearly structurally different from other FGFRs since, unlike other FGFRs, FGFRL1 lacks the tyrosine kinase domain, which is a signaling region in the cell (Non-patent Document 4). Further, the functional aspect of FGFRL1 is also different from other FGFRs, and FGFRL1 has been considered to act to suppress the cell growth (Non-patent Document 5). Accordingly, FGFRL1 is a molecule clearly distinguishable from other FGFRs, and should be clearly distinguished from the FGFRs also in view of its biological role and the industrial applicability deduced therefrom.

[Non-patent Document 1] International Journal of Molecular Medicine 16, 1169-1173, 2005

[Non-patent Document 2] Molecular Cell 35, 856-867, 2009.

[Non-patent Document 3] Journal of Biological Chemistry 286, 420-428, 2011

[Non-patent Document 4] Genomics 69, 275-279, 2000

[Non-patent Document 5] Journal of Biological Chemistry 278, 33857-33865, 2003

### SUMMARY OF THE INVENTION

### Problems to be Solved by the Invention

An object of the present invention is to provide a novel therapeutic agent for cancer such as esophageal squamous cell carcinoma. Another object of the present invention is to provide a method for predicting the prognosis of cancer such as esophageal squamous cell carcinoma. Still another object of the present invention is to provide a method for detecting, or predicting the prognosis of, cancer such as esophageal squamous cell carcinoma using a sample that can be collected less invasively.

As described specifically in the Examples below, the present inventors collected esophageal squamous cell carcinoma tissues from a large number of esophageal squamous cell carcinoma patients to investigate expression of FGFRL1, and conducted a follow-up study on association of the expression level with the prognosis. As a result, it was found that high expression of FGFRL1 is associated with poor prognosis. Further, the present inventors discovered that allowing an anti-FGFRL1 antibody to act on esophageal squamous cell carcinoma cells enables suppression of the growth of the cancer cells.

That is, the present invention provides a therapeutic agent for cancer, comprising as an effective component an antibody that undergoes antigen-antibody reaction with FGFRL1 to suppress the growth of cancer cells, or an antigen-binding fragment thereof Further, the present invention provides a therapeutic method for cancer, comprising administering to a cancer patient an effective amount of an antibody that undergoes antigen-antibody reaction with FGFRL1 to suppress the growth of cancer cells, or an antigen-binding fragment thereof Further, the present invention provides a method for predicting the prognosis of cancer, comprising investigating the expression level of FGFRL1 in a cancer tissue separated from a living body, wherein a high expression level of FGFRL1 indicates poor prognosis. Further, the present invention provides a method for detecting cancer, comprising measuring FGFRL1 or a fragment thereof extracted from a body tissue, or FGFRL1 or a fragment thereof in blood separated from a living body, wherein a higher concentration of FGFRL1 or the fragment thereof contained therein than the concentration of FGFRL1 or the fragment thereof in the tissue or blood of a healthy individual indicates the presence of cancer. Further, the present invention provides a method for predicting the prognosis of cancer, comprising measuring FGFRLI or a fragment thereof in a tissue or blood separated from a cancer patient, wherein a high concentration of FGFRL1 or the fragment thereof contained therein indicates poor prognosis.

### Effect of the Invention

By the present invention, a novel therapeutic agent for cancer such as esophageal squamous cell carcinoma was provided. Further, by the present invention, a novel method for predicting the prognosis of cancer such as esophageal squamous cell carcinoma was provided. Since this method enables prediction of the prognosis of a highly malignant cancer such as esophageal squamous cell carcinoma, and hence enables appropriate selection of a therapeutic method, the method contributes to treatment of cancer. Further, by the present invention, a method for detecting a cancer such as esophageal squamous cell carcinoma using a sample that can be collected less invasively was provided. Since this method is less invasive, the burden of the subject is light. Therefore, detection of cancer can be easily achieved also in medical examination and the like, and early detection and early treatment of cancer are possible.

### BRIEF DESCRIPTION OF THE DRAWINGS

FIG. 1 shows photographs for comparison of the results of immunohistochemistry of esophageal squamous cell carcinoma carried out in the Examples below with the results obtained for normal tissues.

FIG. 2 is a diagram showing the relationship between the expression level of FGFRL1 in the esophageal squamous cell carcinoma tissue measured in the Examples below and the survival rate of patients at each month.

FIG. 3 is a diagram showing the results of measurement of the growth capacity of esophageal squamous cell carcinoma cells observed after allowing an anti-FGFRL1 antibody to act on the cells in the Examples below, as compared to the results obtained by treatment with a control antibody.

### BEST MODE FOR CARRYING OUT THE INVENTION

As described above, the therapeutic agent for cancer of the present invention comprises as an effective component an antibody that undergoes antigen-antibody reaction with FGFRL1 or an antigen-binding fragment thereof Both the amino acid sequence and the gene base sequence are known for FGFRL1. A base sequence of cDNA of FGFRL1 and the amino acid sequence encoded thereby are described, for example, as NCBI Accession NO. NM_001004356.2. The base sequence of cDNA of the FGFRL1 gene and the amino acid sequence encoded thereby are shown in SEQ ID NO:1, and the amino acid sequence alone is shown in SEQ ID NO:2. FGFRL1 is a single-transmembrane protein.

The antibody used herein is an antibody that suppresses the growth of cancer cells, and the antibody undergoes antigen-antibody reaction with the N-terminal region of FGFRL1, that is, the epitope of the antibody is preferably present in the N-terminal region of FGFRL1 or in a region comprising the whole or a part of the N-terminal region (that is, a region that extends from the N-terminal region to another region). The N-terminal region herein means the extracellular region of FGFRL1, that is, the region between the N-terminus and the 378th amino acid in the amino acid sequence shown in SEQ ID NO:2. The antibody may be either a polyclonal antibody or a monoclonal antibody. A polyclonal antibody whose epitope is the N-terminal region of FGFRL1 is also commercially available, and such a commercially available product may also be preferably used. Further, an antigen-binding fragment of the above-described antibody, such as the Fab fragment or the F(ab′)₂ fragment, may also be used. Whether or not the antibody or fragment has an effect to suppress the growth of cancer cells can be investigated by, for example, using the well-known WST1 method as described in the Examples below.

The antibody may be an antibody prepared by genetic engineering, or may be a humanized antibody prepared by replacing the Fc region with that of a human antibody for suppression of rejection reaction in human. Further, in antibody preparations, those prepared by binding a polyethylene glycol (PEG) chain or the like to an end of an antibody for making the antibody less likely to be degraded by protease in the living body are widely used. Also in the therapeutic agent for cancer of the present invention, a stabilizing structure such as a PEG chain may be attached to an end of the above-described antibody or the antigen-binding fragment thereof, and the resultant may be contained in its entirety in the agent as an effective component. In cases where the antibody or an antigen-binding fragment thereof is stabilized by PEGylation, the size of the PEG is several thousand to 50,000, preferably about 10,000 to 50,000. The method for binding PEG to an end of a polypeptide is well known. Such a product prepared by attaching a stabilizing structure is also included in the “antibody or an antigen-binding fragment thereof” in the present invention.

Further, the present antibody may also be utilized as a complex antitumor agent by chemically binding a low molecular weight antitumor agent or a compound having cytotoxicity against cancer thereto, or may be utilized as a navigator in a drug delivery system (DDS) to cancer cells

In terms of the administration route of the therapeutic agent for cancer of the present invention, either parenteral administration or oral administration may be carried out. Parenteral administration such as injection to the cancer tissue, intravenous injection, or intramuscular injection is preferred. The dose may be appropriately set depending on the clinical condition and the severity of the disease to be treated. For example, the therapeutic agent is administered at a dose of 0.1 to 20 mg per administration, preferably 1 to 10 mg per administration, per kg body weight. Further, the therapeutic agent for cancer of the present invention may be formulated by a well-known method into, for example, a solution in which the agent is dissolved in a physiological buffer. Further, a known additive(s) may be added to the solution.

Examples of the cancer to be treated with the therapeutic agent for cancer of the present invention include, but are not limited to, epithelial solid cancers. Esophageal squamous cell carcinoma is especially preferred.

The present inventors discovered, as specifically described in the Examples below, that the expression level of FGFRL1 in a cancer tissue can be used as an index for predicting the prognosis of the cancer, that is, the survival rate after initiation of cancer treatment. Thus, the present invention also provides a method for predicting the prognosis of cancer, which method comprises investigating the expression level of FGFRL1 in a cancer tissue separated from a living body, wherein a high expression level of FGFRL1 indicates poor prognosis. The expression level of FGFRL1 can be measured by an immunoassay such as immunohistochemistry. For the immunoassay, the above-described anti-FGFRL1 antibody or an antigen-binding fragment thereof may be used, and a polyclonal antibody or monoclonal antibody whose epitope is the extracellular region of FGFRL1 may be preferably used. Since, as described above, such a polyclonal antibody is also commercially available, it is also possible to use a commercially available product. Since FGFRLI is expressed in a state where it is penetrating the membrane, the immunohistochemical staining as described in the Examples below is preferably carried out as the immunoassay, but the immunoassay is not limited thereto.

The higher the expression level of FGFRL1, the poorer the prognosis may be. Thus, by preliminarily investigating the expression level of FGFRL1 and the prognosis in a large number of patients with the same kind of cancer, it is possible to predict the prognosis based on how high the expression level of FGFRL1 is. For example, as specifically described in the Examples below, in cases where the expression level is investigated by immunohistochemical staining, the prediction may be made based on evaluation of the stained area per cancer tissue (0%: 1-50%: +, 51-100%: ++), and the positivity per cell wherein strong positivity is evaluated as (+++) and negativity is evaluated as (−). In consideration of the extent of expression of FGFRLI in a normal tissue, a total value of not less than (++) can be judged as positive (that is, poor prognosis).

As described above, FGFRL1 is a single-transmembrane protein, and has a structure that undergoes the action of protease in the extracellular region. Therefore, it is thought that a tissue fluid extracted from a tissue, or blood, may contain free FGFRL 1 or a free fragment of FGFRL 1. The present inventors inferred that, by quantifying free FGFRL1 or a free fragment of FGFRL1 in a tissue fluid extracted from a tissue, or blood, cancer can be detected. The present invention also provides a method for detecting cancer, which method comprises measuring FGFRLI or a fragment thereof extracted from a body tissue, or FGFRL1 or a fragment thereof in blood separated from a living body, wherein a higher concentration of FGFRLI or the fragment thereof contained therein than the concentration of FGFRLI or the fragment thereof in the tissue or blood of a healthy individual indicates the presence of cancer. In such cases, the FGFRL1 or a fragment thereof in the tissue extract or blood can be quantified by an immunoassay using an antibody that undergoes antigen-antibody reaction with the extracellular region of FGFRL1. The immunoassay in such cases may be carried out by a well-known method such as ELISA, which is widely used for quantification of various proteins in body fluid; the sandwich method, in which a fluorescent label or chemiluminescent label is used; or the immunoagglutination method, in which sensitized particles prepared by immobilizing an antibody on latex particles are used. The cut-off value in such cases may be a value significantly different from the mean value in healthy individuals. The cut-off value may be, for example, 1.0 unit/mL, and the unit value in such cases is determined using as a standard the concentration in the tissue or blood of a healthy individual, although the unit value may vary depending on differential diagnosis from similar diseases and on background factors of the patient.

Further, based on the abundance of FGFRL1 or a fragment thereof in a tissue fluid extracted from a tissue, or blood, prediction of the prognosis of cancer can be carried out similarly to the above-described cases where the prediction is carried out based on the expression level of FGFRL1 in the cancer tissue. In such a case, the criteria for evaluation of the prognosis may vary depending on whether the survival rate or the recurrence rate is to be evaluated, and for what disease the evaluation is to be done.

The present invention is described below more specifically by way of Examples. However, the present invention is not limited to the Examples below.

### EXAMPLES

### Example 1

### Immunohistochemical Staining

Tissue sections were prepared from esophageal squamous cell carcinoma tissues collected from 69 esophageal squamous cell carcinoma patients, and subjected to deparaffinization (3 times of 5 minutes of immersion in xylene, 2 times of 3 minutes of immersion in 100% ethanol, 3 minutes of immersion in 95% ethanol, 3 minutes of immersion in 90% ethanol, 3 minutes of immersion in 85% ethanol, 5 minutes of washing with running water, and then 5 minutes of immersion in distilled water) and then antigen retrieval by heat treatment (treatment in 1 mM Tris buffer (pH 9.0) supplemented with 0.1 mM EDTA at 95° C. for 40 minutes, followed by allowing the resultant to cool at room temperature for 20 minutes, washing with running water and then immersion in distilled water). Subsequently, endogenous peroxidase was blocked with 3% hydrogen peroxide solution (at room temperature for 10 minutes), and the sections were then washed with distilled water 3 times, followed by immersion in 5 mM Tris buffer (pH 7.2) supplemented with 0.005% Tween 20 and 15 mM NaCl at room temperature for 5 minutes for achieving equilibration. Anti-FGFRL1 antibody H-300 (Santa cruse) was 50-fold diluted with Dako REAL antibody diluent (Dako), and treatment was carried out with the resulting dilution for 30 minutes. After washing the sections with 5 mM Tris buffer (pH 7.2) supplemented with 0.005% Tween 20 (trade name) and 15 mM NaCl 3 times, coloring with DAB (diaminobenzidine) was performed using Dako ChemMate ENVISION kit (Dako), followed by washing with distilled water and then performing counter staining with Dako REAL Hematoxylin (prepared by 4-fold dilution with distilled water and then addition of Tween 20 (trade name) to adjust the Tween 20 concentration to 0.01%) at room temperature for 3 minutes. Thereafter, washing with water, dehydration, clearing and embedding were carried out (5 minutes of washing with water, 5 minutes of immersion in 80% ethanol, 5 minutes of immersion in 90% ethanol, 5 minutes of immersion in 95% ethanol, 2 times of 5 minutes of immersion in 100% ethanol, and 3 times of 5 minutes of immersion in xylene, followed by embedding with Leica CV5030). The stained area per cancer tissue (0%: 0, 1-50%: 1, 51-100%: 2) and the staining intensity (no signal: 0, weak: 1, moderate: 2, marked: 3) were scored, and a total score of not less than 4 was defined as high expression of the FGFRL1 protein, and a total score of less than 4 was defined as low expression of the FGFRL1 protein. The survival rate was compared between both groups of patients by the Kaplan-Meier method and the log-rank test.

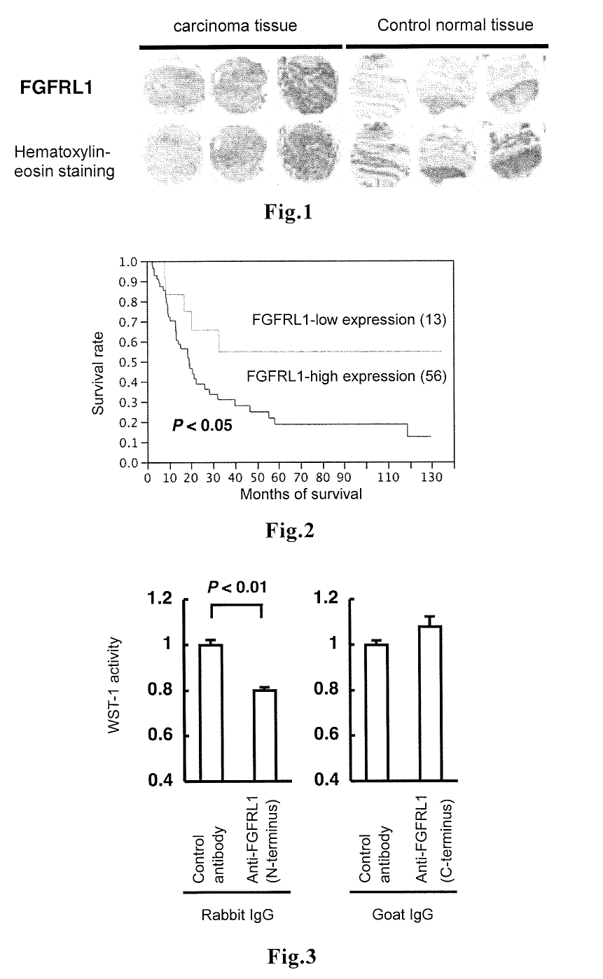

The results of immunohistochemical staining are shown in FIG. 1, and the relationship between the expression level of FGFRL1 and the survival rate of patients at each month is shown in FIG. 2. As is evident from FIG. 2, the prognosis was poor in the cases where the expression level of FGFRL1 was high, and the survival rate at Month 60 in these cases was a little more than one third of the survival rate observed in the cases where the expression level of FGFRL1 was low.

### Example 2

### Pharmacological Effect

An esophageal squamous cell carcinoma-derived cell line KYSE-170 was plated in Ham F12 (Nissui)/RPMI1640 (Gibco) medium (pH 6.8) supplemented with fetal bovine serum (5%, Equitech-Bio) filtered through a 0.22-μm PVDF membrane filter (Millipore), penicillin (100 unit/ml, Meiji), gentacin (4.44 mg/l, Schering) and sodium hydrogen carbonate (0.2%) on a 96-well dish (5×10³ cells/100 μL/well), and cultured under the conditions of 5% CO₂, a humidity of 100% and a temperature of 37° C. Twenty four hours later, the cells were treated with anti-FGFRL1 antibody H-300 (recognition site: N-terminus/extracellular region) or C-20 (recognition site: C-terminus/intracellular region) (Santa cruse) and a control IgG (Santa cruse) of the animal from which it was derived, which were diluted with the above-described Ham F12/RPMI1640 medium (final concentration, 20 μg/ml ). After 24 hours of culture, the cell growth was evaluated by the well-known WST1 method using a commercially available reagent.

The results are shown in FIG. 3. As shown in FIG. 3, in the case where the monoclonal antibody whose epitope is the N-terminal region, that is, the extracellular region, of FGFRL1 was used, the growth of esophageal squamous cell carcinoma cells was significantly suppressed as compared to the case where the treatment was carried out with the control antibody. Thus, such an antibody is useful as a therapeutic agent for esophageal squamous cell carcinoma.

### [SEQUENCE LISTING]

1. A method to suppress FGFRL1 expressing cancer cell growth, said method comprising administering to FGFRL1 expressing cancer cells an effective amount of an antibody that binds to the N-terminal region of FGFRL1 to suppress growth of FGFRL1 expressing cancer cells, or an antigen-binding fragment thereof, wherein the N-terminal region of FGFRL1 is the extracellular region of FGRFRL1 and the extracellular region of FGFRL1 is the region between the N-terminus and the 378^{th} amino acid of SEQ ID NO: 2.

2. The method according to claim 1, wherein said FGFRL1 expressing cancer cells are esophageal squamous cell carcinoma cells.

3. The method according to claim 1, wherein the FGFRL1 expressing cancer cells are KYSE 170 cells.

4. The method according to claim 3, wherein the antibody or an antigen-binding fragment thereof is H-300.

5. A therapeutic method for FGFRL1 expressing cancer, said therapeutic method comprising administering to a cancer patient an effective amount of an antibody that binds to the N-terminal region of FGFRL1 to suppress growth of the cancer cells, or an antigen-binding fragment thereof, wherein the N-terminal region of FGFRL1 is the extracellular region of FGRFRL1 and the extracellular region of FGFRL1 is the region between the N-terminus and the 378^{th} amino acid of SEQ ID NO: 2.

6. The method according to claim 1, wherein the antibody or an antigen binding fragment thereof is an IgG antibody.

7. The method according to claim 1, wherein the antibody or an antigen binding fragment thereof is a polyclonal antibody.

8. The method according to claim 1, wherein the antibody or an antigen binding fragment thereof is a monoclonal antibody.

9. The method according to claim 1, wherein the antibody or antigen binding fragment thereof is a humanized antibody.

10. The method according to claim 1, wherein the antibody or antigen-binding fragment thereof is bound to a low molecular weight antitumor agent or a compound having cytotoxicity against cancer thereto.

11. The method according to claim 1, wherein the antibody or antigen-binding fragment is administered via a patient orally.

12. The method according to claim 1, wherein the antibody or antigen binding fragment is administered via a patient parentally.

13. The method according to claim 1, wherein the amount administered via a patient at a dose of 0.1 to 20 mg per administration, per kg body weight.

14. The method according to claim 1, wherein the amount administered via a patient at a dose of 1 to 10 mg per administration, per kg body weight.

15. The method according to claim 5, wherein said FGFRL1 expressing cancer is esophageal squamous cell carcinoma.

16. The method according to claim 5, wherein the antibody or antigen binding fragment thereof is a humanized antibody.

17. The method according to claim 5, wherein the antibody or antigen-binding fragment is administered orally.

18. The method according to claim 5, wherein the antibody or antigen binding fragment is administered parentally.

19. The method according to claim 5, wherein the amount administered is at a dose of 0.1 to 20 mg per administration, per kg body weight.

20. The method according to claim 5, wherein the amount administered is at a dose of 1 to 10 mg per administration, per kg body weight.
